(19) United States
(12) Patent Application Publication
(10) Pub. No.: US 2005/0222543 A1
(43) Pub. Date: Oct. 6, 2005

(54) Skin-care protective gloves and manufacturing method

(76) Inventor: Zechuan Shao, Santa Clara, CA (US)

Correspondence Address: Donald W. Meeker;Patent Agent, 924 East Ocean Front, #E, San Francisco, CA, 92661, US

(21) Appl. No.: 10/817,390

(22) Filed: Apr. 1, 2004

(51) Int. Cl. A61M 35/00

(52) U.S. Cl. 604/292

(57) ABSTRACT

Protective natural rubber and synthetic elastomeric gloves are disclosed which have an inner skin-contacting absorbent layer. The inner layer is impregnated with skin care compositions. During application of the gloves, the inner layer absorbs perspiration from the hand and readily and slowly releases skin care compositions to soothe and condition the hand. Methods of manufacture are described.

### BACKGROUND OF THE INVENTION

1. Field of the Invention

This invention relates generally to protective natural rubber and synthetic elastomeric perspiration-absorbing gloves which have an inner skin-contacting absorbent layer impregnated with skin care compositions so that during application of the gloves, the inner layer absorbs perspiration from the hand and readily and slowly releases skin care compositions to soothe and condition the hand.

2. Description of the Prior Art

Rubber gloves usually create an unhealthy environment for the hands. They can cause skin irritations from the hot interior of the gloves. They normally make the hands sweat. Prior art attempts to improve the situation have not produced an effective solution.

Prior art U.S. Pat. No. 6,274,154 issued Aug. 14, 2001 to Chou discloses a moisturizing and therapeutic glove which includes a thin layer of Aloe Vera coated evenly and uniformly on an inside surface of the glove. Aloe Vera is attached to the surface through a dehydration process achieved with a controlled drying method. Aloe Vera soothes hand during the wearing of the glove.

Prior art U.S. Pat. No. 6,423,328 issued Jul. 23, 2002 to Chou also discloses a moisturizing and therapeutic glove which includes a thin layer of Aloe Vera coated evenly and uniformly on an inside surface of the glove. Aloe Vera is attached to the surface through a dehydration process achieved with a controlled drying method. Aloe Vera soothes hand during the wearing of the glove.

Prior art U.S. patent application # 20010048937, published Dec. 6, 2001 by Chou, shows a moisturizing and therapeutic glove which includes a thin layer of Aloe Vera coated evenly and uniformly on an inside surface of the glove. Aloe Vera is attached to the surface through a dehydration process achieved with a controlled drying method. Aloe Vera soothes hand during the wearing of the glove.

Prior art U.S. patent application # 20020025335, published Feb. 28, 2002 by Chou, also shows a protective glove includes a coating of dehydrated material on its inside surface. The dehydrated material, in contact with perspiration from a hand wearing the glove, soothes the hand. Some methods of placing the coating onto the inside surface of the glove include spraying or dipping with a solution that includes Aloe Vera.

Prior art U.S. patent application # 20020110584, published Aug. 15, 2002 by Chou, also shows a moisturizing and therapeutic glove which includes a thin layer of Aloe Vera coated evenly and uniformly on an inside surface of the glove. Aloe Vera is attached to the surface through a dehydration process achieved with a controlled drying method. Aloe Vera soothes the hand during the wearing of the glove.

Prior art U.S. patent application # 20030017193, published Jan. 23, 2003 by Chou, illustrates a protective glove includes a coating of dehydrated material on its inside surface. The dehydrated material, in contact with perspiration from a hand wearing the glove, soothes the hand. Some methods of placing the coating onto the inside surface of the glove include spraying or dipping with a solution that includes Aloe Vera.

The disadvantages of the methods claimed in Chou's patents include:

Spraying or dipping in solution is not suitable for gloves made of non-impregnating, non porous, non-absorbing material like latex and other synthetic materials. Since the solution tends to flow in the direction of the force of gravity on the slippery surface of the gloves, most of the solution will be lost before drying.

There is no guarantee that an effective amount of skin care compositions will remain on the inner surface of the glove as most of the solution is lost before drying.

There are waste concerns as most of the solution runs off the glove surface before drying.

Exposing Aloe Vera and other skin care compositions in a water-based solution to the environment for an extended period, can cause decomposition and loss of effectiveness.

Production is costly.

Other prior art patents also fail to adequately solve the problem.

Prior art U.S. patent application # 20020114825, published Aug. 22, 1002 by Leong, puts forth a method for manufacturing, a thin-walled, skin-contacting elastomeric article to be worn over a human body part, wherein an article-shaped form is dipped into a latex composition that has been mixed with the extract of the plant Aloe Vera forming the article. The article is processed and cured so that the extract is present within the article as one or combination of a polymer, co-polymer and filler of the article. An Aloe Vera coating may be applied to at least one surface of the article.

Prior art U.S. Pat. No. 4,853,978, issued Aug. 8, 1989 to Stockum, describes an antimicrobial medical glove consisting essentially of an outer elastomeric body in the shape of a hand and an inner coating containing an antimicrobial agent, said inner coating being capable of slowly releasing said antimicrobial agent in an amount and over a period of time sufficient to maintain an essentially bacteria-free and fungus-free environment within said glove after said glove has been donned.

Prior art U.S. Pat. No. 5,614,202, issued Mar. 25, 1997 to DeFina, illustrates a moisturizing glove in which a middle layer saturated with lotion, an exterior layer of non-porous material formed to the top side of the middle layer, and an inner layer having a plurality of pores, formed to the bottom side of the middle layer create a cavity for receiving and enveloping a human extremity, particularly a human hand.

Prior art U.S. patent application # 20010048936, published Dec. 6, 2001 Prenovitz, details a device for dispensing a skin treating agent on an appendage and its method of manufacture. The device having a first side with an inner surface and an outer surface, a second side with an inner surface and an outer surface, the first side and second side connected at an outer seal with the inner surfaces facing one another and forming a chamber for receiving and enclosing the appendage. The first and second sides having a perforated area which when torn apart creates an opening where the appendage is inserted into the chamber and a quantity of skin treating agent distributed in discrete areas on at least one of the inner surfaces of the chamber. A securing member enables the device to be secured to the appendage.

Prior art U.S. Pat. No. 2,916,036, issued Dec. 8, 1959 to Sutton, discloses an article of personal wear, such as a glove, comprising a body of unguent-free rubber-like material, a surface layer of rubber-like material and globules of an unguent material, distributed within said rubber-like material, a layer of fibers adherent to said surface layer and which, when the article is worn, will be next to the skin of the user and at least some of said fibers extending into said surface layer and into contact with at least some of the globules of said unguent material to provide paths for the movement of unguent material out of said surface layer.

Prior art U.S. Pat. No. 3,896,807, issued Jul. 29, 1975 to Buchalter, concerns an article to place in contact with the body for use in applying a cosmetic or therapeutic substance to the skin, comprising a substrate and a therapeutic or cosmetic composition carried by said substrate and comprising the oil phase of a cream formulation, said oil phase being a dry emulsifiable solid, which upon addition of moisture thereto forms an oil in water or water in oil emulsion.

Prior art U.S. Pat. No. 4,476,588, issued Oct. 16, 1984 to Long, claims a disposable hand care product comprising first and second glove side elements fabricated of a relatively thin plastic film material, each of these elements having oppositely extending finger receiving areas and juxtapositioned palm receiving areas, these side elements being disposed in face-to-face aligned relationship and being sealingly interconnected to one another around the entire periphery thereof so as to define a pair of glove members arranged in wrist-to-wrist connected relationship, a hand care product disposed interjacent these side elements, and separating means permitting convenient separation of the pair of glove members from one another and simultaneously providing access into the interior of the member so that a person can insert his or her hands into these members and thereby have skin contact with the hand care product disposed therewithin.

Prior art U.S. Pat. No. 4,775,372, issued Oct. 4, 1988 to Wilberg, describes a device for application of liquids to the surface of hands which permits slow release of the liquid on the surface of the hands and at the same time permits one to continue to work with their hands. The device is shaped like a glove or mitt prepared from a thin flexible sheet material possessing a plurality of spaced enclosed small pouches containing the liquid to be applied to the hands, the glove or mitt also having a wrist band which fits tightly around the wrist to prevent the treatment liquid from running onto the wrist while the gloves are in place. The pouches are either punctured prior to placing the glove on the hand, or are burst by application of external pressure on the device after being placed on the hand.

Prior art U.S. Pat. No. 5,335,373, issued Aug. 9, 1994 to Dangman, et al., is for a protective medical glove containing a liquid antiseptic composition and methods for its use. The flexible glove comprises at least a thin inner layer and at least a thin outer layer of material; the outer layer is preferably the more elastic and less plastic layer. Between the layers of the glove, a liquid antiseptic composition is stored which comprises an antiseptic in a liquid. The liquid antiseptic composition may also contain a surface-active agent, an algesic agent, a colorant, a vasoconstrictive agent, a smell-causing chemical, and a viscosity-modifying agent. A glove puncture by an object may cause some transfer of liquid antiseptic composition from the glove onto the hand and into a hand wound should the wound occur; useful as an immediate treatment to help to prevent a possible systemic infection by a pathogen in the individual; the treatment can help to protect a gloved individual such as a surgeon, a medical doctor, a health care worker or another worker whose work may place them at risk of becoming contaminated by a glove-puncturing object contaminated with the AIDS virus, hepatitus B virus or another infectious pathogen.

Prior art U.S. Pat. No. 5,357,636, issued Oct. 25, 1994 to Dresdner, Jr., et al., discloses a flexible protective medical glove containing a non-liquid antiseptic composition and methods for its use. The glove comprises a thin inner layer and a thin outer layer of material; preferably the outer layer is a more elastic and less plastic layer than the inner layer. A compartment between the layers of the glove is capable of providing a non-liquid antiseptic composition which comprises an antiseptic in a non-liquid composition. The non-liquid antiseptic composition may also contain a surface-active agent, an algesic agent, a colorant, a vasoconstrictive agent, an odorant, or a viscosity-modifying agent. An object puncturing the glove wall can become coated with the non-liquid antiseptic composition and can automatically transfer some of the antiseptic composition from the glove onto the hand and into a hand wound should the object cause a wound; useful as an immediate preventative antiseptic treatment to help to decontaminate the hand and hand wound of infectious pathogens that may have been transferred there by the object. The treatment can help to protect a gloved individual such as a surgeon, a medical doctor, a health care worker, a law enforcement officer, a dentist or any worker whose work may place them at some risk of becoming contaminated through the hands by an infectious pathogen including the AIDS virus or hepatitis B virus.

Prior art U.S. Pat. No. 4,881,277, issued Nov. 21, 1989 to Hogle, illustrates a protective glove for protection against infection due to puncture or rupture of the glove during surgery formed from two protective layers. The outer layer is formed with substantially the same finger lengths as the inner layer and slightly larger diametrical width than the inner layer. This forms a protective clearance space while providing tactile sensitivity to allow fine surgical techniques to be performed.

Prior art U.S. Pat. No. 5,459,880, issued Oct. 24, 1995 to Sakaki, et. al., claims a glove that has, at least partially, a laminated part in which a plurality of different types of rubbers are successively laminated. In the laminated part, the lowermost layer is a natural rubber layer, and the top surface layer is an oil-resistant rubber layer (for example, an acrylonitrile-butadiene rubber layer). The glove manufacturing method according to the present invention comprises the steps of immersing a glove mold in natural rubber latex containing a heat-sensitive coagulant to form a coating of the lowermost layer on the mold surface, and immersing at least a part of the lowermost layer in another rubber latex different from the first-mentioned rubber latex. The glove of the present invention has high strength and is economical and excellent in oil resistance and solvent resistance.

Prior art U.S. Pat. No. 5,794,266, issued Aug. 18, 1998 Han, shows a glove made with thin latex or rubber material that is suitable for carrying out medical or other delicate procedures while maintaining comfort by creating air pockets inside the glove. The inner layer of the glove has projections or granules so that the contact surface between the glove and the hand is reduced. The glove has a plurality of convexed projections formed on the inner surface so that the convexed projections are in contact with the hand and creates air pockets or buffers in the immediate vicinity of the convexed projections. The glove can be easily made by using a mold having concaved indents or spots and dipping the mold into a pool of liquid latex or rubber and then peeling the glove off of the mold. Each finger tip of the glove may be devoid of the convexed projections to enhance sensitivity.

Prior art U.S. Pat. No. 5,869,072, issued Feb. 9, 1999 to Berry, describes a therapeutic applique for treatment of dry hands, either as a patch, face mask, or glove. The applique has the form of a porous, flexible sheet that is applied to the skin, and the sheet has a water-activatable material carried on its surface and also permeating its pores. The water-activated material, when in a dry, inactive state, preferably has a moisture content of less than 10% by weight, of the sheet, and is soluble in water. The arrangement is such that when the applique is applied to the skin, along with water, the applique's water-soluble component partially disassociates from it and leaches onto the skin, either while the sheet is in place on the skin or prior to the sheet being placed on the skin. In addition, with time the outermost layer of the water-soluble component undergoes evaporation, causing it to partially re-solidify, thereby forming an occlusive outer barrier which tends to retain the remaining water-soluble components between itself and the skin. There results improved, all natural skin moisturization, without preservatives and without undesirable greasy or oily residue being left on the skin.

Prior art U.S. Pat. No. 5,965,276, issued Oct. 12, 1999 to Shlenker, et al., illustrates single and multiple layer membranes such as gloves and condoms that include one or more deactivating barrier layers and/or indicating layers to indicate to a user membrane breach or the presence of a harmful substance in blood or body fluids. A membrane may include one or more permeable or semipermeable layers to disperse contained substances such as lubricants, biocides, spermicides, or indicators outwardly, and may also include permeable or semipermeable layers to allow transmission of body fluids or other environmental fluids inwardly into contact with an indicating or treating substance. An intermediate layer of a multi-layer membrane may include a substance to wipe, cleanse, sterilize, or otherwise treat a piercing needle. A membrane may include a sealing or coating to entrap indicators or other agents such as biocides therein. A method of making membranes such as gloves results in a double glove having discrete inner or outer layers joined only in a cuff region. Admixing of gentian violet with latex prior to membrane formation provides biocidal properties, anti-aging effects prolonging shelf-life and tear resistance, and reduces allergic reactions in latex-allergic users.

Prior art U.S. Pat. No. 6,117,119, issued Sep. 12, 2000 to Gould, puts forth a gelatinous body protection article having a therapeutic additive, wherein a vitamin additive such as Vitamin A, B.sub. 12, C, D, E, is incorporated into the thermoplastic material of a sock, glove or like body protection article. The thermoplastic material is preferably a block copolymer such as SEBS, SEPS and SEEPS copolymer. Additionally, the thermoplastic material can include natural oils such as grape seed oil, avocado oil, jojoba oil, canola oil, ceramides and aloe.

Prior art U.S. Pat. No. 6,299,817, issued Oct. 9, 2001 to Parkinson, claims a process which allows elastomeric materials of different colors, durometers and/or other material characteristics to chemically bond to one another so as to eliminate any possibility of delamination. Latex-based liquid elastomer solutions having different material characteristics are applied sequentially to a heated mold to form a series of layers making up the article, with rapid partial curing taking place between each application; the partial curing forms a skin coat over each layer which prevents intermixing of the different solutions, but which still permits a chemical bond to form during full curing of the materials. The process applies equally to dip (immersion) molded and flat molded goods. An example of the invention as it relates to flat-molded goods is an athletic shoe sole, and an example as it relates to dip-molded goods is a resilient, one piece watersports boot which can be fitted over a human foot without a zipper.

Prior art U.S. Pat. No. 6,488,948, issued Dec. 3, 2002 to Danieli, relates to a water-based composition having at least one of anti-bacterial or anti-fungal properties and use thereof for skin care and for fabric treatment. The composition is not only effective as an anti-bacterial and/or anti-fungal material when included in a skin care gel or lotion for topical use, such as in a sun screen composition, or in conjunction with sanitary elastic gloves as a coating therein, it is gentle to the user's skin and may include constituents which advantageously indicate its presence. The composition is not only effective as an anti-bacterial and/or anti-fungal material when used as a liquid to treat fabric employed for personal hygiene aids including disposable diapers for children and adults, sanitary napkins, and wipes, it is gentle to the skin and may be formulated to provide odor control. Users include humans and animals.

Prior art U.S. Pat. No. 6,618,861, issued Sep. 16, 2003 to Saks, et al., discloses a method of multiple dipping to make gloves with a clear wrist portion, which allows glove wearers to see through the transparent wrist portion and tell time with the gloves on. The method is applicable to all the common materials to make gloves, or coat gloves, via dipping: including natural rubber latex, nitrile rubber latex(carboxylated or non-carboxylated), polychloroprene latex, polyisoprene latex, polyurethane latex, polyvinyl chloride and their blends, or mixtures thereof. The invention provides procedures to make compounds, which are both transparent and opaque, as well as dipping sequences for the materials. Products, with the added feature of a transparent wrist portion, are provided without sacrificing performance and without increasing production cost.

What is needed is protective natural rubber and synthetic elastomeric perspiration-absorbing gloves which have an inner skin-contacting absorbent layer impregnated with skin care compositions so that during application of the gloves, the inner layer of each glove absorbs perspiration from the hand and readily and slowly releases skin care compositions to soothe and condition the hand, which gloves have a non-fiber perspiration-absorbing inner layer, and which gloves are fabricated by a method which alleviates concerns of waste of the skin care compositions, and which method applies an effective concentration of skin care compositions, such as Aloe Vera, evenly distributed over the inner layer of a disposable glove, and which method minimizes exposure to the environment of the skin care compositions in a water based solution, avoiding decomposition and loss of effectiveness of the skin care compositions.

### SUMMARY OF THE INVENTION

An object of the present invention is to provide protective natural rubber and synthetic elastomeric perspiration-absorbing gloves which have an inner skin-contacting absorbent layer impregnated with skin care compositions so that during application of the gloves, the inner layer of each glove absorbs perspiration from the hand and readily and slowly releases skin care compositions to soothe and condition the hand.

Another object of the present invention to provide a protective glove with a non-fiber perspiration-absorbing inner layer.

A further object of the present invention is to provide a disposable protective glove impregnated with skin care compositions, including Aloe Vera, which alleviates concerns of waste of the skin care compositions.

One more object of the present invention is to provide an improved method for applying an effective concentration of skin care compositions, such as Aloe Vera, to the inner layer of a disposable glove.

An additional object of the present invention is to provide an improved method of minimizing exposure to the environment of the skin care compositions in a water based solution, avoiding decomposition and loss of effectiveness of the skin care compositions.

In brief, the present invention is a novel disposable glove with at least two layers, including an inner skin-contacting layer coated with at least one of the following: Aloe Vera, Vitamin E, Vitamin C, Peppermint powder, Grape Seed Extract, and Cucumber Extract. The glove provides a skin protective barrier with a therapeutic and/or cosmetic benefit. The glove also minimizes the abrasion between the hand and inner layer, resulting in less skin irritation. When in the moist environment of perspiration on the hand of the wearer, the glove releases the skin-care compositions and transfers them to the skin by normal contact, thereby maintaining and/or improving the skin health of the wearer.

Aloe Vera has been widely used for the treatment of various skin conditions for thousands of years. Studies have shown that aloe Vera facilitates the healing of wounds and damaged skin tissue. It also exerts anti-fungal and antibacterial effects. It has moisturizing and pain relieving properties for the skin lesions, in addition to healing effects. Aloe Vera also prevents skin damage resulting from exposure to ultraviolet radiation associated with direct sunlight.

Aloe Vera is a frequent moisturizing ingredient in cosmetics and hair care products. Aloe-containing creams, gels, and ointments are used for minor burns, sunburn, cuts, and abrasions, wounds, and skin irritations. Aloe is also found in many shampoos, soaps, suncreens, skin creams and other cosmetics that are used to soothe, heal, protect, and moisturize the skin.

Vitamin E is well known as an antioxidant responsible for proper functioning of the immune system and for maintaining health skin. It helps keep skin smooth and soft, improves skin moisture by offsetting dryness caused by aging and environmental factors. Natural water soluble Vitamin E (d alpha tocopherol), is derived from natural sources. Studies have shown that it is more easy to be absorbed by skin and more effective than the synthetic version, usually made from petrochemicals.

Vitamin E is a potent antioxidant by itself, but its effectiveness is magnified when applied with other antioxidants, especially with Vitamin C.

Vitamin C has been found to be an effective ingredient in skin care treatments. Vitamin C acts as a major anti-oxidant and anti-inflammatory. Antioxidants fight against oxygen free radicals, which are stimulated by exposure to UV light, tobacco smoke, and other environmental insults. Those oxygen free radicals can damage and destroy the skin.

Grape seed extract is the primary commercial source of a group of powerful antioxidants known as oligomeric proanthocyanidins (OPCs). Studies have indicated OPCs are much more effective than vitamin C and vitamin E in neutralizing free oxygen radicals, which contribute to organ degeneration and aging in humans. Grape seed extract is a mixture of complex compounds and has wide range of therapeutic uses, its antioxidant properties are believed to help slow the aging process. One of the extract's most popular uses is in treating the affects of aging, including preventing wrinkles by protecting the skin against ultraviolet radiation damage from sunburn, improving skin elasticity and tone, and helping reduce the appearance of scars and stretch marks.

Peppermint (Mentha piperita) is a natural hybrid of water mint (Mentha aquatica) and spearmint (mentha spicata). Peppermint is a cooling, relaxing herb that contains properties that help ease inflamed tissues, calm muscle spasms or cramps, and inhibit bacteria and microorganisms. It also has pain-relieving and infection preventing qualities.

Cucumber Extract has also been found to have beneficial effects on the skin.

An advantage of the present invention is to soothe and condition the wearer's hands during application of the gloves by including an inner skin-contacting layer coated with Aloe Vera, Vitamin E, Vitamin C, Peppermint powder, Grape Seed Extract, and/or Cucumber Extract.

Another advantage of the present invention is that the inner layer is made of absorbing material that helps the glove retain the skin care compositions.

An additional advantage of the present invention is that it can guarantee an effective amount of skin care compositions contact the skin of the wearer as the amounts can be easily controlled during manufacture.

One more advantage of the present invention is that there are no waste concerns since the skin care compositions are applied onto the wet inner surface of the gloves.

An additional advantage of the present invention is that it minimizes the exposure time of the skin care compositions to the environment to only a few minutes, avoiding decomposition and loss of effectiveness of the skin care compositions.

Still another advantage of the present invention is that it provides an improved disposable glove which is comfortable to wear over an extended period of time, and also economical and easy to manufacture.

### BRIEF DESCRIPTION OF THE DRAWINGS

These and other details of my invention will be described in connection with the accompanying drawings, which are furnished only by way of illustration and not in limitation of the invention, and in which drawings:

FIG. 1 is a diagrammatic view of a flow chart showing the manufacturing method of the present invention without chlorination;

FIG. 2 is a diagrammatic view of a flow chart showing the manufacturing method of the present invention with chlorination;

FIG. 3 is a diagrammatic view of a flow chart showing the manufacturing method of the present invention without hydrogel coating;

FIG. 4 is a side elevational cross-sectional view through a powder applying tumbler drum.

### BEST MODE FOR CARRYING OUT THE INVENTION

A disposable protective natural rubber or synthetic elastomeric perspiration-absorbing glove comprises at least one skin care layer, an inner facing layer contacting the skin. The inner facing layer is coated or impregnated with moisturizing and therapeutic skin care compositions. The skin care compositions can be released in a moist environment by perspiration on the hand of the wearer. The skin care compositions can be transferred to the wearer's skin by normal contact. The disposable elastomeric glove further comprises at least one outer facing layer, which serves the normal functions of a glove relative to protecting the wearer in medical, scientific, health care, maintenance, or other professions where protecting the hands from contact with hazardous materials.

The inner facing layer is coated by an impregnating of the layer with a solution of Aloe Vera, Vitamin E, Vitamin C, Grape Seed Extract, Peppermint and/or Cucumber applied to the glove interior in powder form, by methods preferably such as blow/spraying the solutions onto the gloves while the gloves are tumbled in a drum. The solution is within the range of approximately 5 wt. % to approximately 30 wt. % Aloe Vera, approximately 5 wt % to approximately 15 wt. % water soluble Vitamin E, approximately 1 wt % to approximately 10 wt % Grape Seed Extract, approximately 1% wt to approximately 10 wt. % Vitamin C, and approximately 1 wt. % to approximately 3 wt. % Peppermint. No water is added, just powder applied to the wet interior surface of the gloves.

The inner skin-contacting layer is coextensive with the outer layer and is a non-water repellent layer made of non-fiber absorbent material, hydrogel, or synthetic hydrophilic material. The skin-contacting layer is preferably formed for a propenoic acid based hydrogel and has an elongation to break greater than 200%.

The disposable glove comprises two layers; one outer facing layer and an inner facing skin care layer.

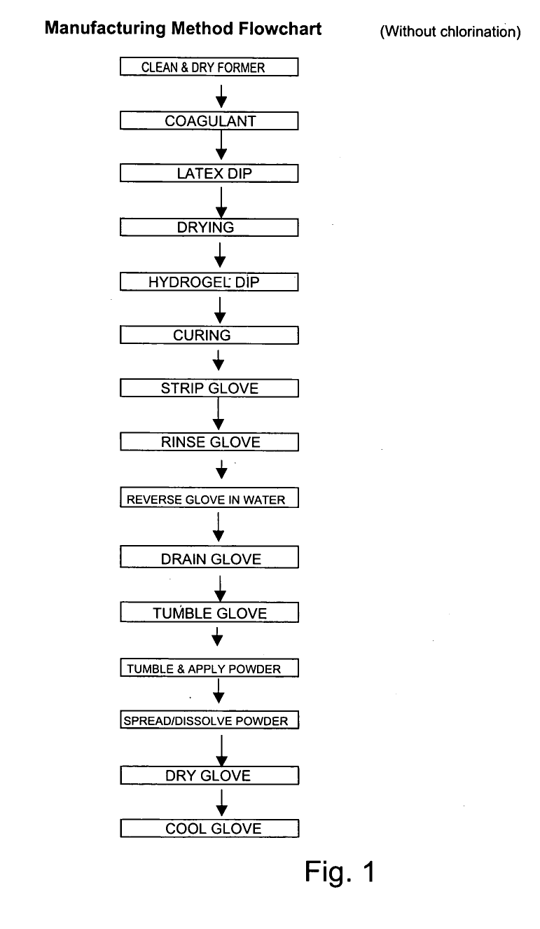

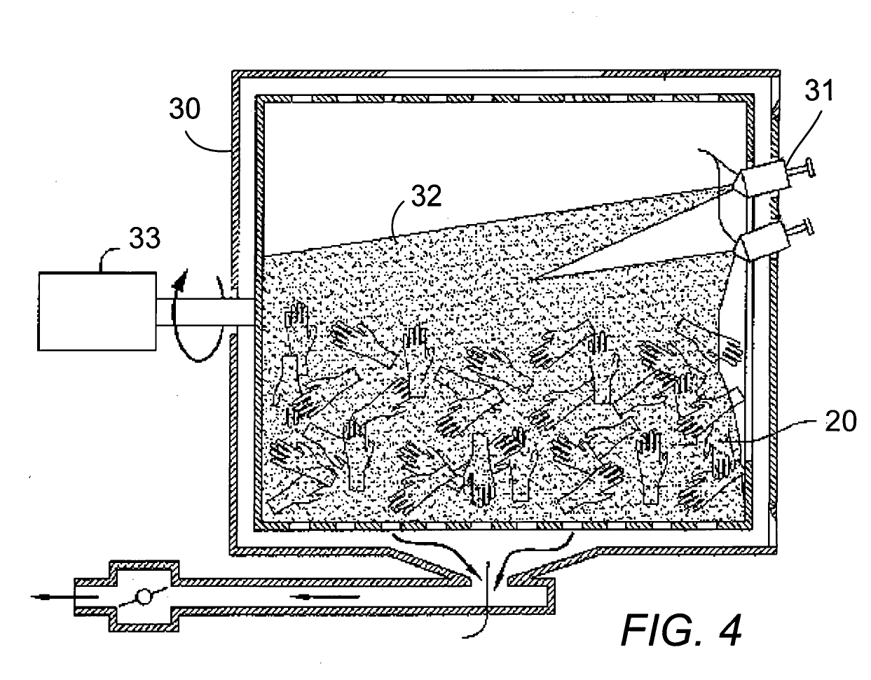

The method of manufacture of the disposable glove comprises the following steps, as shown in FIG. 1:
a) dipping a glove mold into the compounded natural rubber or synthetic latex to form a first layer of a glove on the mold (latex dip);
b) leaching and drying the first layer of the glove on the mold (drying);
c) dipping the first layer of a glove on the mold into a pre-prepared dispersion of a liquid absorbing material, such as hydrogel with solidifying agents, to form a second layer of a glove (hydrogel dip);
d) heating the double layers on the mold in an oven to cure and solidify the first and second layers of the glove on the mold (curing);
e) removing the mold from the oven and stripping the coated glove from the mold (strip glove);
f) rinsing the glove with water to remove the powder and chemical residues (rinse glove);
g) reversing the glove to turn the glove inside out whereby the inner surface of the glove faces outward (reverse glove in water);
h) draining the reversed glove in an extractor to remove excess water, while the inner layer of the glove is still wet, and leaving the glove's cuff area closed (drain glove);
i) placing the wet glove in a speed-controlled drum of a tumbling apparatus equipped with a metering pump and at least one blow/spray nozzle (tumble glove);
j) tumbling the gloves in the speed-controlled drum 30, in FIG. 4, at a speed such that the glove remains in the bottom half of the drum (tumble and apply powder);
k) during tumbling actuated by a motor 33, the metering pump moves the at least one skin care composition 32 into and through the at least one blow nozzle 31 to evenly apply skin care compositions onto the inner surface of the glove 20 (spread/dissolve powder—ideally, application of the skin care compositions is performed intermittently in at least four iterations of at least 5-10 seconds in duration each);
l) removing the glove from the tumbling apparatus into a dryer;
m) drying the glove at a temperature of 55 degrees C. until completely dried (dry glove); and
n) cooling the glove to room temperature (cool glove).

The inner layer of the glove continues to absorb the skin care compositions during drying.

The glove may be further on-line or off-line chlorinated

The glove may be made either by an automatic production line or manual production line.

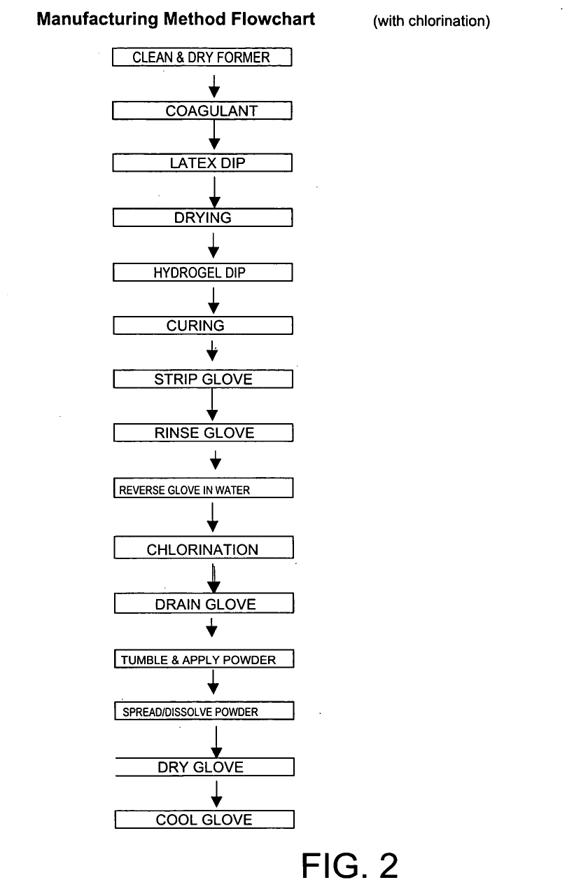

The method including described above including hydrogel coating may have an additional step of chlorination between reversing the glove in water (g) and draining the glove (h) as shown in FIG. 2.

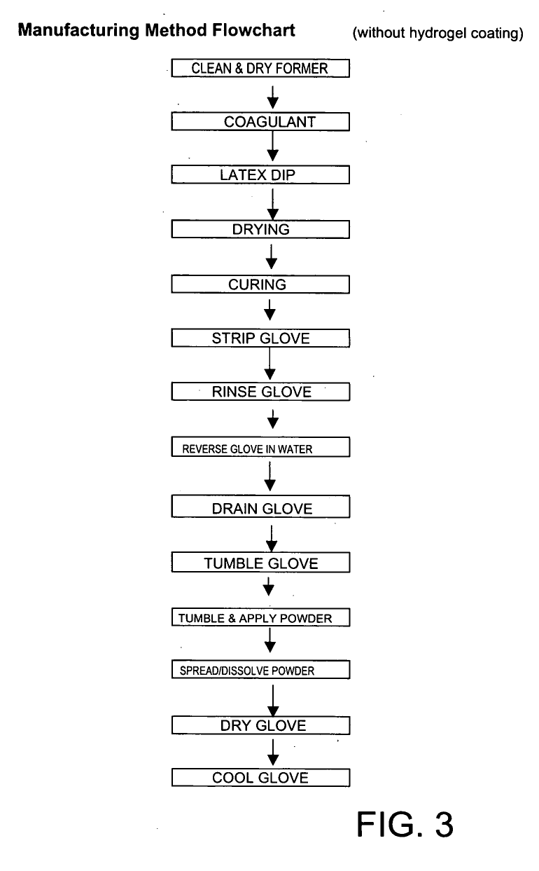

An alternate method, as shown in FIG. 3, describes the manufacturing method of applying powder formed of skin care compositions directly onto the inner surface of the glove without hydrogel coating, as follows:

A method of manufacturing a disposable protective natural rubber and synthetic elastomeric glove with the inner surface of the glove coated with at least one therapeutic and moisturizing skin care composition by directly applying powder formed of at least one therapeutic and moisturizing skin care composition onto the inner surface of the glove, the method comprising:
the first step of forming at least one outer facing layer by dipping and drying a glove mold in a liquid rubber-type material and drying it;
the second step of heating the glove to dry the glove, removing the glove from mold, rinsing the glove with liquid, and turning the glove inside out still wet;
the third step of rinsing the glove with water to remove the powder and chemical residues, reversing the glove to turn the glove inside out whereby the inner surface of the glove faces outward;
the forth step of draining the reversed glove in an extractor to remove excess water, while the inner layer of the glove is still wet, and leaving the glove's cuff area closed;
the fifth step of placing the wet glove in a speed-controlled drum of a tumbling apparatus equipped with a metering pump and at least one blow/spray nozzle, tumbling the gloves in the speed-controlled drum at a speed such that the glove remains in the bottom half of the drum during tumbling, the metering pump moves the at least one powder formed skin care composition into and through the at least one blow nozzle to evenly apply skin care compositions onto the inner surface of the glove, allowing the powder to be spread and dissolved on the glove inner surface during tumbling;
the sixth step of removing the glove from the tumbling apparatus into a dryer, drying the glove to have skin care compositions form a coating on the inner surface of the glove;
the seventh step of cooling the glove to room temperature.

It is understood that the preceding description is given merely by way of illustration and not in limitation of the invention and that various modifications may be made thereto without departing from the spirit of the invention as claimed.

1. A disposable protective multiple layer natural rubber and synthetic elastomeric glove comprising:
an inner skin-contacting absorbent layer evenly coated over the entire skin-contacting absorbent layer with at least one therapeutic and moisturizing skin care composition absorbed into the skin-contacting layer as an evenly sprayed powder absorbed into the skin-contacting absorbent layer when wet, the moisturizing skin contacting composition impregnating the skin-contacting absorbent layer and dried, the skin-contacting absorbent layer adapted to absorb perspiration from a hand of a wearer and the at least one skin care composition adapted for being released in a moist environment by perspiration and the at least one skin care composition adapted for transfer to skin on a hand of a wearer by normal contact; and
at least one outer facing rubber-type material layer.

2. The glove of claim 1 wherein the inner layer is a non-fiber absorbent material.

3. The glove of claim 1 wherein the inner layer is a synthetic hydrophilic material.

4. The glove of claim 1 wherein the inner layer is a propenoic acid based hydrogel.

5. The glove of claim 1 wherein the inner layer has an elongation to break greater than 200%.

6. The glove of claim 1 wherein the inner layer is impregnated with a solution comprising at least one skin care composition selected from the group of water soluble skin care composition substances including Aloe Vera, Vitamin E, Vitamin C, Peppermint powder, Grape seed Extract, and Cucumber extract.

7. The glove of claim 1 wherein the outer layer of the glove is made of natural rubber latex.

8. The glove of claim 1 wherein the outer layer of the glove is made of acrylonitrile.

9. The glove of claim 1 wherein the outer layer of the glove is made of polyvinylchloride.

10. The glove of claim 1 wherein the outer layer of the glove is made of polyurethane.

11. The glove of claim 1 wherein the outer layer of the glove is made of polybutadiene.

12. A method of manufacturing a disposable protective multiple layer natural rubber and synthetic elastomeric glove with an inner skin-contacting absorbent layer coated with at least one therapeutic and moisturizing skin care composition impregnating the skin care layer, the method comprising:
the first step of forming at least one outer facing layer by dipping and drying a glove mold in a liquid rubber-type material and drying it;
the second step of forming an inner skin-contacting absorbent layer by dipping the glove mold into a liquid absorbent material over the rubber-type material, heating the glove to dry the two layers, removing the glove mold, rinsing the glove with liquid, and turning the glove inside out still wet; and
the third step of spraying a powder formed of at least one therapeutic and moisturizing skin care composition evenly over the entire wet skin-contacting absorbent layer and allowing the powder to be absorbed into the wet skin-contacting absorbent layer impregnating the skin-contacting absorbent layer and drying the skin-contacting absorbent layer while the powder is still being absorbed, the skin-contacting absorbent layer adapted for absorbing perspiration from a hand of a wearer and the at least one skin care composition adapted for being released in a moist environment by perspiration and the at least one skin care composition adapted for transfer to skin on a hand of a wearer by normal contact.

13. The method of claim 12 wherein the first step comprises dipping the glove mold into a compounded natural rubber or synthetic latex to form the outer facing layer of a glove on the mold and leaching and drying the outer facing layer of the glove on the mold.

14. The method of claim 12 wherein the second step comprises dipping the outer facing layer of the glove on the mold into a pre-prepared dispersion of hydrogel with solidifying agents to form the inner skin-contacting absorbent layer of the glove, heating the two layers on the glove mold in an oven to cure and solidify the two layers of the glove on the mold, removing the glove mold from the oven, stripping the coated glove from the glove mold, rinsing the glove with water to remove the powder and chemical residues, reversing the glove to turn the glove inside out whereby the inner skin-contacting absorbent layer of the glove faces outward, and draining the reversed glove in an extractor to remove excess water, while the inner layer of the glove is still wet, and leaving a cuff area of the glove closed.

15. The method of claim 14, wherein the third step further comprises placing the wet glove in a speed-controlled drum of a tumbling apparatus equipped with a metering pump and at least one blow/spray nozzle, tumbling the gloves in the speed-controlled drum at a speed such that the glove remains in the bottom half of the drum, and during tumbling, operating a metering pump to move the at least one skin care composition into and through the at least one blow nozzle to apply skin care compositions evenly onto the inner surface of the glove.

16. The method of claim 15, wherein the skin care compositions applied are in a water soluble powder form.

17. The method of claim 15, wherein the application of the skin care compositions is performed intermittently in at least four iterations of at least 5-10 seconds in duration each.

18. The method of claim 16, wherein the powder is applied onto the inner layer of the glove by direct contact on the glove and by transference, whereby gloves rub against each other to spread the powder during tumbling.

19. The method of claim 16, wherein the powder applied onto the glove is dissolved and at least partially absorbed by the wet inner layer of the glove.

20. The method of 15, further comprising the steps of removing the glove from the tumbling apparatus into a dryer, drying the glove at a temperature of 55 degrees C. until completely dried, and cooling the glove to room temperature.

21. The method of claim 20, wherein the inner layer of the glove continues to absorb the skin care compositions during drying.

22. The method of claim 12 wherein the glove may be further on-line or off-line chlorinated.

23. The method of claim 12 wherein the glove can be made either by an automatic production line or manual production line.

24. A method of manufacturing a disposable protective natural rubber and synthetic elastomeric glove with the inner surface of the glove coated with at least one therapeutic and moisturizing skin care composition by directly applying powder formed of at least one therapeutic and moisturizing skin care composition onto the inner surface of the glove, the method comprising:
the first step of forming at least one outer facing layer by dipping and drying a glove mold in a liquid rubber-type material and drying it;
the second step of heating the glove to dry the glove, removing the glove from mold, rinsing the glove with liquid, and turning the glove inside out still wet;
the third step of rinsing the glove with water to remove the powder and chemical residues, reversing the glove to turn the glove inside out whereby the inner surface of the glove faces outward;
the forth step of draining the reversed glove in an extractor to remove excess water, while the inner layer of the glove is still wet, and leaving the glove's cuff area closed;
the fifth step of placing the wet glove in a speed-controlled drum of a tumbling apparatus equipped with a metering pump and at least one blow/spray nozzle, tumbling the gloves in the speed-controlled drum at a speed such that the glove remains in the bottom half of the drum during tumbling, the metering pump moves the at least one powder formed skin care composition into and through the at least one blow nozzle to evenly apply skin care compositions onto the inner surface of the glove, allowing the powder to be spread and dissolved on the glove inner surface during tumbling;
the sixth step of removing the glove from the tumbling apparatus into a dryer, drying the glove to have skin care compositions form a coating on the inner surface of the glove;
the seventh step of cooling the glove to room temperature.
